(19) United States
(12) Patent Application Publication
(10) Pub. No.: US 2003/0184294 A1
(43) Pub. Date: Oct. 2, 2003

(54) Multiple channel, neuro vascular array coil for magnetic resonance imaging

(76) Inventors: Eddy Benjamin Boskamp, Menomonee, WI (US); John E. Lorbiecki, Hubertus, WI (US); LeRoy Raymond Blawat II, Milwaukee, WI (US)

Correspondence Address: CANTOR COLBURN, LLP, 55 GRIFFIN ROAD SOUTH, BLOOMFIELD, CT, 06002

(21) Appl. No.: 10/063,226

(22) Filed: Apr. 1, 2002

(51) Int. Cl. G01V 3/00; A61B 5/05

(52) U.S. Cl. 324/318; 324/309; 600/411

(57) ABSTRACT

A multiple channel array coil for magnetic resonance imaging is disclosed. In an exemplary embodiment, the array coil includes a cylindrically tapered head portion having a plurality of individual coil elements. A chest portion further includes a generally planar anterior section and a generally planar posterior section, with both the anterior section and said posterior section including a plurality of individual coil elements.

### BACKGROUND OF INVENTION

The present disclosure relates generally to magnetic resonance imaging (MRI) systems and, more particularly, to a multiple channel, neurovascular array coil for MRI.

A conventional MRI device establishes a homogenous magnetic field, for example, along an axis of a person's body that is to undergo MRI. This homogeneous magnetic field conditions the interior of the person's body for imaging by aligning the nuclear spins of nuclei (in atoms and molecules forming the body tissue) along the axis of the magnetic field. If the orientation of the nuclear spin is perturbed out of alignment with the magnetic field, the nuclei attempt to realign their nuclear spins with an axis of the magnetic field. Perturbation of the orientation of nuclear spins may be caused by application of radio frequency (RF) pulses. During the realignment process, the nuclei precess about the axis of the magnetic field and emit electromagnetic signals that may be detected by one or more coils placed on or about the person.

The frequency of the nuclear magnetic radiation (NMR) signal emitted by a given precessing nucleus depends on the strength of the magnetic field at the nucleus' location. As is well known in the art, it is possible to distinguish radiation originating from different locations within the person's body simply by applying a field gradient to the magnetic field across the person's body. For the sake of convenience, direction of this field gradient may be referred to as the left-to-right direction. Radiation of a particular frequency may be assumed to originate at a given position within the field gradient, and hence at a given left-to-right position within the person's body. The application of such a field gradient is also referred to as frequency encoding.

However, the simple application of a field gradient does not allow for two-dimensional resolution, since all nuclei at a given left-to-right position experience the same field strength, and hence emit radiation of the same frequency. Accordingly, the application of a frequency-encoding gradient, by itself, does not make it possible to discern radiation originating from the top versus radiation originating from the bottom of the person at a given left-to-right position. Resolution has been found to be possible in this second direction by application of gradients of varied strength in a perpendicular direction to thereby perturb the nuclei in varied amounts. The application of such additional gradients is also referred to as phase encoding.

Frequency-encoded data sensed by the coils during a phase encoding step is stored as a line of data in a data matrix known as the k-space matrix. Multiple phase encoding steps are performed in order to fill the multiple lines of the k-space matrix. An image may be generated from this matrix by performing a Fourier transformation of the matrix to convert this frequency information to spatial information representing the distribution of nuclear spins or density of nuclei of the image material.

MRI has proven to be a valuable clinical diagnostic tool for a wide range of organ systems and pathophysiologic processes. Both anatomic and functional information can be gleaned from the data, and new applications continue to develop as the technology and techniques for filling the k-space matrix improve. As technological advances have improved achievable spatial resolution, for example, increasingly finer anatomic details have been able to be imaged and evaluated using MRI. Often, however, there is a tradeoff between spatial resolution and imaging time, since higher resolution images require a longer acquisition time. This balance between spatial and temporal resolution is particularly important in cardiac MRI, for example, where fine details of coronary artery anatomy must be discerned on the surface of a rapidly beating heart.

Imaging time is largely a factor of desired signal-to-noise ration (SNR) and the speed with which the MRI device can fill the k-space matrix. In conventional MRI, the k-space matrix is filled one line at a time. Although many improvements have been made in this general area, the speed with which the k-space matrix may be filled is limited.

To overcome these inherent limits, several techniques have been developed to simultaneously acquire multiple lines of data for each application of a magnetic field gradient. These techniques, which may collectively be characterized as “parallel imaging techniques”, use spatial information from arrays of RF detector coils to substitute for the encoding which would otherwise have to be obtained in a sequential fashion using field gradients and RF pulses. The use of multiple effective detectors has been shown to multiply imaging speed, without increasing gradient switching rates or RF power deposition.

One such parallel imaging technique that has recently been developed and applied to in vivo imaging is referred to as SENSE (SENSitivity Encoding). The SENSE technique is based on the recognition of the fact that the spatial sensitivity profile of the receiving elements (e.g., resonators, coils, antennae) impresses on the spin resonance signal position information that can be used for the image reconstruction. The parallel use of a plurality of separate receiving elements, with each element having a different respective sensitivity profile, and combination of the respective spin resonance signals detected enables a reduction of the acquisition time required for an image (in comparison with conventional Fourier image reconstruction) by a factor which in the most favorable case equals the number of the receiving members used (see Pruessmann et al., Magnetic Resonance in Medicine Vol. 42, p.952-962, 1999).

A drawback of the SENSE technique, however, results when the component coil sensitivities are either insufficiently well characterized or insufficiently distinct from one another. These instabilities may manifest as localized artifacts in the reconstructed image, or may result in degraded SNR. Accordingly, it is desirable to implement RF coil arrays in MRI systems that (among other aspects) provide increased SNR with or without the use of parallel imaging techniques such as SENSE.

### SUMMARY OF INVENTION

The above discussed and other drawbacks and deficiencies of the prior art are overcome or alleviated by a multiple channel array coil for magnetic resonance imaging. In an exemplary embodiment, the array coil includes a cylindrically tapered head portion having a plurality of individual coil elements. A chest portion further includes a generally planar anterior section and a generally planar posterior section, with both the anterior section and said posterior section including a plurality of individual coil elements.

In another aspect, a multiple channel array coil for magnetic resonance imaging has a cylindrically tapered head portion with a plurality of individual coil elements. A chest portion has a generally planar anterior section and a generally planar posterior section, both the anterior section and the posterior section including a plurality of individual coil elements. In addition, a hinge assembly enables the anterior section of the chest portion to be rotated about a left-right axis and translated in a vertical axis of the array coil.

In still another aspect, a magnetic resonance imaging (MRI) system, includes a computer, a magnet assembly for generating a polarizing magnetic field, and a gradient coil assembly for applying gradient waveforms to the polarizing magnetic field along selected gradient axes. In addition, a radio frequency (RF) transceiver system is used for applying RF energy to excite nuclear spins of an object to be imaged, and for thereafter detecting signals generated by excited nuclei of the object to be imaged. The RF transceiver system further includes a multiple channel array coil having a cylindrically tapered head portion and a chest portion. The head portion includes a plurality of individual coil elements, and the chest portion has a generally planar anterior section and a generally planar posterior section. Both the anterior section and the posterior section include a plurality of individual coil elements. The signals detected by the multiple channel array coil are processed by the computer to produce MR images of the object to be imaged.

In yet another aspect, a method for configuring a multiple channel array coil suitable for use in sensitivity encoding for magnetic resonance imaging (MRI) includes arranging a first set of individual coil elements into a cylindrically tapered head portion. A second and a third set of individual coil elements are arranged into a chest portion, the chest portion having a generally planar anterior section, including the second set of individual coil elements, and a generally planar posterior section including the third set of individual coil elements.

Finally, in still a further aspect, method for implementing sensitivity encoding for magnetic resonance imaging (MRI) includes generating a polarizing magnetic field and applying gradient waveforms to the polarizing magnetic field along selected gradient axes. RF energy generated by an RF transceiver system is then applied to excite nuclear spins of an object to be imaged, and thereafter signals generated by excited nuclei of the object to be imaged are detected. The RF transceiver system further includes a first set of individual coil elements arranged into a cylindrically tapered head portion, and a second and a third set of individual coil elements arranged into a chest portion. The chest portion further includes a generally planar anterior section including the second set of individual coil elements, and a generally planar posterior section including the third set of individual coil elements.

### BRIEF DESCRIPTION OF DRAWINGS

Referring to the exemplary drawings wherein like elements are numbered alike in the several Figures:

FIG. 1 is a schematic block diagram of an exemplary MR imaging system suitable for use with the present invention embodiments;

FIG. 2 is a perspective view of a multiple channel, neurovascular phased array coil suitable for SENSE imaging, in accordance with an embodiment of the invention;

FIG. 3 is a perspective view of the array coil of FIG. 2, further illustrating a mounting substrate and hinge assembly;

FIG. 4 is a sectional view of part of the chest portion of the array coil, illustrating transformer isolation of the individual coil elements;

FIG. 5 is a sectional view of part of the head portion of the array coil, illustrating transformer isolation of the individual coil elements;

FIGS. 6 and 7 illustrate simulated g-factor maps for the head and chest portions of the array coil, illustrating a comparison between a 10 mm coil separation configuration and a coil overlapping configuration; and

FIG. 8 illustrates simulated g-factor maps for the head and chest portions of the array coil at a reduction factor of 3.

### DETAILED DESCRIPTION

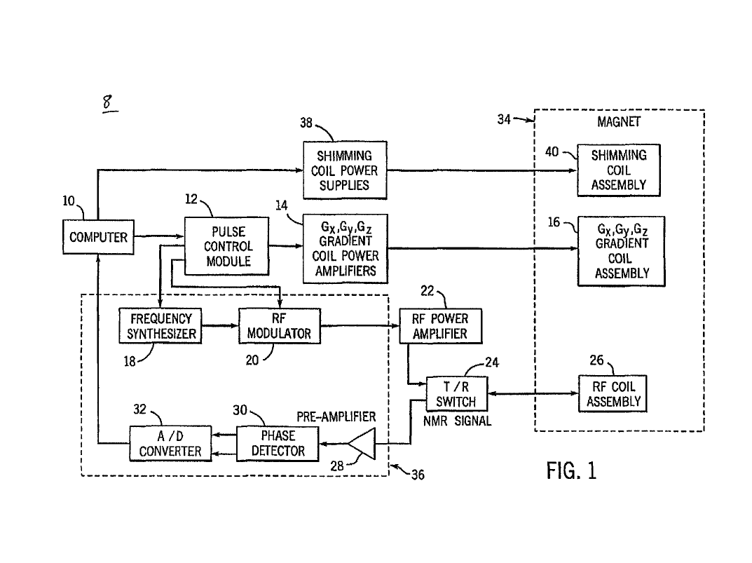

Referring initially to FIG. 1, an exemplary magnetic resonance (MR) imaging system 8 suitable for use with the present invention embodiments includes a computer 10, which controls gradient coil power amplifiers 14 through a pulse control module 12. The pulse control module 12 and the gradient amplifiers 14 together produce the proper gradient waveforms Gx, Gy, and Gz, for either a spin echo, a gradient recalled echo pulse sequence, a fast spin echo, or other type of pulse sequences. The gradient waveforms are connected to gradient coils 16, which are positioned around the bore of an MR magnet assembly 34 so that gradients Gx, Gy, and Gz are impressed along their respective axes on the polarizing magnetic field B₀ from magnet assembly 34.

The pulse control module 12 also controls a radio frequency synthesizer 18 that is part of an RF transceiver system, portions of which are enclosed by dashed line block 36. The pulse control module 12 also controls an RF modulator 20, which modulates the output of the radio frequency synthesizer 18. The resultant RF signals, amplified by power amplifier 22 and applied to RF coil 26 through transmit/receive switch 24, are used to excite the nuclear spins of the imaged object (not shown).

The MR signals from the excited nuclei of the imaged object are picked up by the RF coil 26 and presented to preamplifier 28 through transmit/receive switch 24, to be amplified and then processed by a quadrature phase detector 30. The detected signals are digitized by a high speed A/D converter 32 and applied to computer 10 for processing to produce MR images of the object. Computer 10 also controls shimming coil power supplies 38 to power shimming coil assembly 40.

As stated previously, phased array coils are commonly used in MRI as they offer improved SNR over an extended field of view (FOV). With the advent of parallel imaging techniques, it has also become important to obtain a reliable sensitivity assessment for each individual coil used in conjunction with sensitivity based (SENSE) reconstruction. In addition to the common signal intensity variations, local noise enhancement occurs to varying degrees according to the conditioning of the sensitivity-based reconstruction steps. This effect, which depends strongly upon the geometry of the particular coil arrangement, is quantitatively described by Pruessmann, et al. as the local geometry factor (g).

As will be appreciated, the geometry factor plays a significant role in designing SENSE arrays. The geometry factor is a mathematical function of the coil sensitivities, noise correlation, and the reduction factor R, wherein R denotes the factor by which the number of samples is reduced with respect to conventional, full Fourier encoding. In practice, the coil structure generally does not permit straightforward analytical coil optimization. Thus, simulations have proven to be a valuable tool in seeking optimized coil arrangements for sensitivity encoding, involving the determination of geometry maps and base SNR.

However, it will be appreciated that additional design constraints further dictate that each individual coil within an array be decoupled during the transmit pulse, and that all coils be decoupled from their neighbors during the receive mode so that noise is uncorrelated. Unfortunately, conventional coils with overlap produce very high geometry-related noise enhancement and thus are not suited for SENSE imaging.

Therefore, in accordance with an embodiment of the invention, there is disclosed a multiple channel (e.g., 16-channel), neurovascular phased array coil suitable for SENSE imaging. Although a 16-channel array coil is described hereinafter, it will be appreciated by those skilled in the art that a different number of individual coil elements may be utilized. The phased array coil does not make use of an overlapping coil configuration, and thus mutual coupling between the coils is an inherent characteristic of the device. Accordingly, alternative decoupling techniques that may be used in lieu of overlapping coils include preamplifier decoupling and/or transformer decoupling.

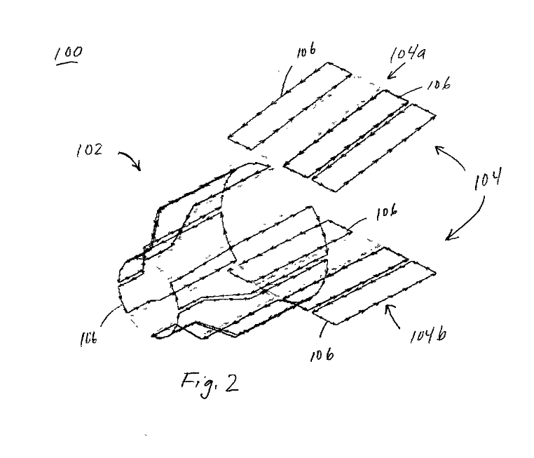

Referring specifically now to FIG. 2, there is shown a perspective view of a multiple channel, neurovascular phased array coil 100 suitable for SENSE imaging. The array coil 100 generally includes a cylindrically tapered, head portion 102 and a chest portion 104 that further includes a generally planar anterior section 104a and posterior section 104b. Although the chest portion section are shown as generally planar in shape, it will be understood that both anterior and posterior sections 104a and 104b may be specifically shaped and/or contoured so as to be in close contact with the body of a patient placed therebetween. Included within the head portion 102 and the anterior and posterior sections 104a, 104b of chest portion 104a are individual, electrically conductive coil elements 106 for receiving RF signals generated by a patient (not shown) during the MR imaging process. In the embodiment depicted, there are eight coil elements 106 comprising the head portion, as well as four coil elements within each of the anterior and posterior sections 104a, 104b of the chest portion 104, for a total of 16 elements or channels in the array. In a preferred embodiment, each coil element 106 is geometrically separated or spaced apart from a nearest neighbor coil without overlap.

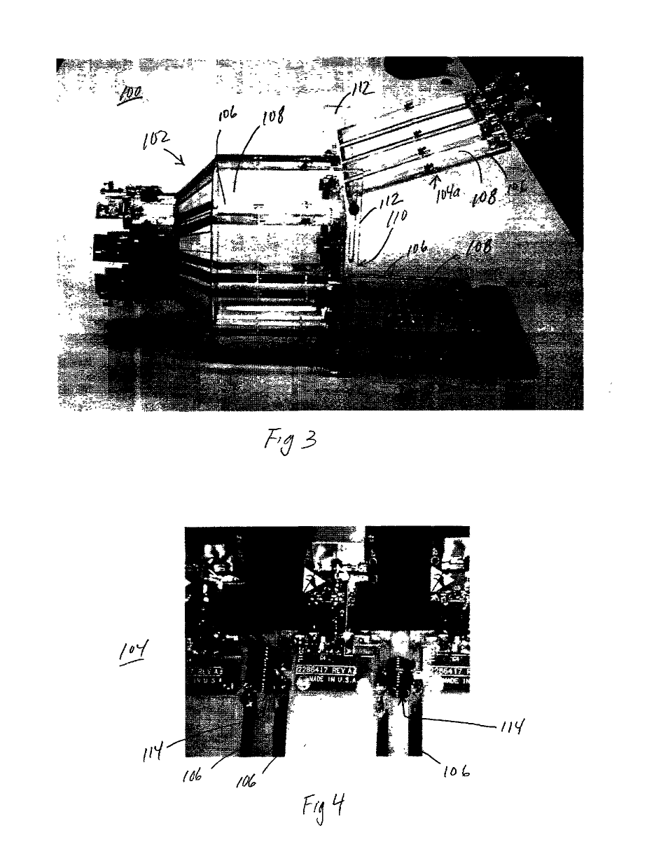

In a further aspect, FIG. 3 illustrates array coil 100 configured within a suitable mounting substrate 108. Most particularly, a hinge assembly 110 includes a pair of slotted arms 112 through which the anterior chest section 104a may be mounted. In this manner, the anterior chest section 104a may be both rotated about a left-right axis and translated in a vertical axis such that close coupling occurs between the individual coil elements 106 of the anterior chest section 104a and the chest of a patient (not shown) for improved SNR.

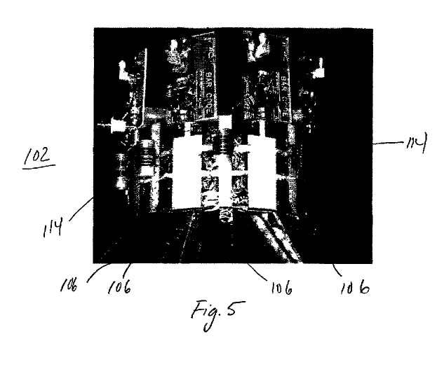

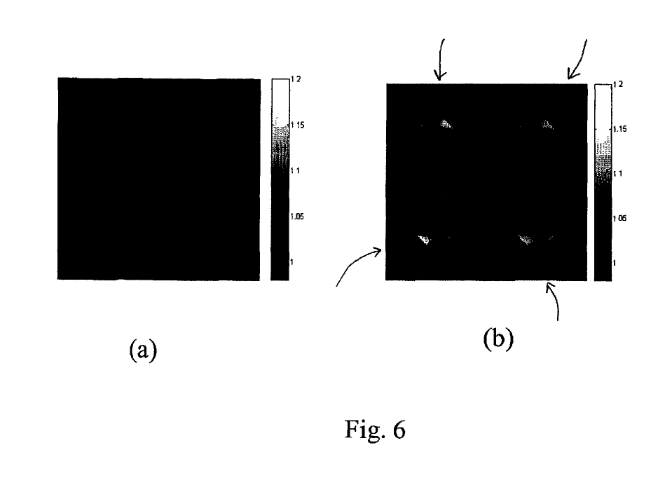

As described earlier, with a non-overlapping coil structure, mutual coupling is inherent characteristic of adjacently spaced coils. When a cluster of close surface coils simultaneously receive signals, the mutual coupling therebetween the coils generates the coupled modes, thereby causing the signal spectrum splitting and resulting in coil detuning. Accordingly, FIGS. 4 and 5 illustrate a transformer decoupling used for coil isolation. In FIG. 4, there is shown a section of one of the chest portions 104 in which one of the individual coils 106 is isolated from its two nearest neighboring coils by transformers 114. Similarly, FIG. 5 illustrates the transformer isolation between coils 106 in the head portion 102, at the tapered end thereof. In addition, the next nearest neighboring coils are decoupled by low impedance preamplifiers, as is described in greater detail later.

In determining the above-described neurovascular coil design, a program was used to calculate geometry factor maps (g-maps) for different array coil configurations. Again, it was found that an array coil with a geometric separation between the individual coil elements results in a far better geometry factor than an array with overlapping elements.

The local SNR of a SENSE image is determined in accordance with the following equation:

$${SNR}^{SENSE} = \frac{{SNR}^{Conventional}}{{gR}^{1 / 4}}$$

wherein SNR^{Conventional} denotes the SNR obtained when the same coil array and imaging scheme are used without reducing the number of phase encoding steps (i.e., in conventional image processing without SENSE techniques), thus requiring the complete scan time. It can be seen, therefore, that for an optimum SNR from SENSE produced images, the geometry factor of the coil should be kept to minimum (the ideal value being 1). Software simulations for the g-maps and B₁ fields were carried out using preexisting routines written in Matlab, again with the main objective being the minimization of the g factor. Coil arrays with different number, shapes, sizes and element orientations were simulated.

The dome geometry of the head portion 102 was selected to reduce the length of the coil and also to optimize homogeneity. Based on the generated g-maps and the B₁ field maps, 8 coils (within head portion 102 ) each having an overall diameter of 265 mm and a length of 327 mm, with 10 mm separation therebetween, were constructed. It was discovered that while larger arrays with smaller diameter coils result in lower g values, smaller coils exhibit lower penetration. Therefore, the total number of the individual coil elements within the head portion 102 was limited to eight as a tradeoff between g factor and coil penetration. The significance of optimized g-maps is demonstrated in FIG. (2) and FIG. (3), wherein it can be seen that a coil-overlapping configuration results in much higher values of g.

The chest portion 104 of the coil 100 was configured with rectangular dimensions of length 253 mm and width 270 mm, with 10 mm separation between the coils. Four of these coils were mounted on the anterior section 104, and four on the posterior section 104b, as shown in FIGS. 2 and 3.

The nearest neighbor coil pairs, decoupled with the transformer method, demonstrated isolations of about −25 dB. Initially, the next nearest coil neighbors were decoupled using 2-ohm input impedance preamplifiers. However, an improved version having coil-integrated preamps with less than 1.5 ohm input impedance is contemplated. The cable lengths were also selected such that the preamp impedance was transferred to the decoupling network, with the impedance from the preamplifier end being 50 ohms (real) for optimum noise figure performance. During RF transmission, the neurovascular array coil was isolated from the body transmit coil by actively switching PIN diodes connected to the coil circuit via parallel resonant tanks.

In a loaded condition, the isolation between adjacent coils was found to be <−25 dB. For sensitivity determination, low resolution reference images were obtained from the SENSE coil. Additional time required for the reference images is not a concern since the time required for the sensitivity determination requires only about 6 seconds.

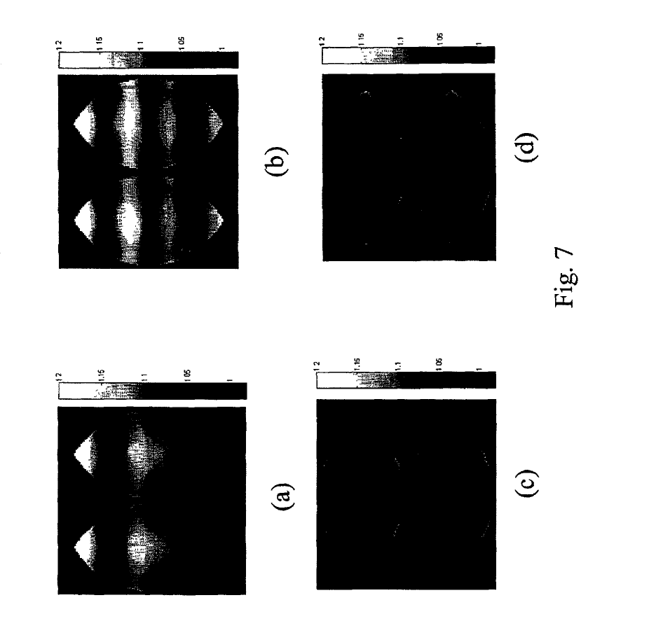

Referring generally now to FIGS. 6 and 7, g-maps were plotted for both the head and chest portions of the coil 100 and compared with those of a reference coil having an overlapping coil configuration (as opposed to the 10 mm separation of the present embodiment). More particularly, FIG. 6 illustrates a simulation of g-maps in SENSE for the head portion 102. In each simulation, the phase encoding is in the horizontal direction. The reduction factor was selected as R=2. Map (a) represents the g-factor plot of the present embodiment with 10 mm coil separation, whereas map (b) denotes corresponding g-factor plot map for the reference coil having a 10 mm coil overlap. As can be seen, the arrows in map (b) illustrate areas of higher values of g.

Similarly, FIG. 7 illustrates a simulation of g-maps for the chest portion the chest portion 104, again with a reduction factor of R=2. Maps (a) and (c) show the present coils with 10 mm separation, while maps (b) and (d) show the overlapped coil simulation. The phase encoding is in the horizontal direction in maps (a) and (b), and in vertical direction in maps (c) and (d).

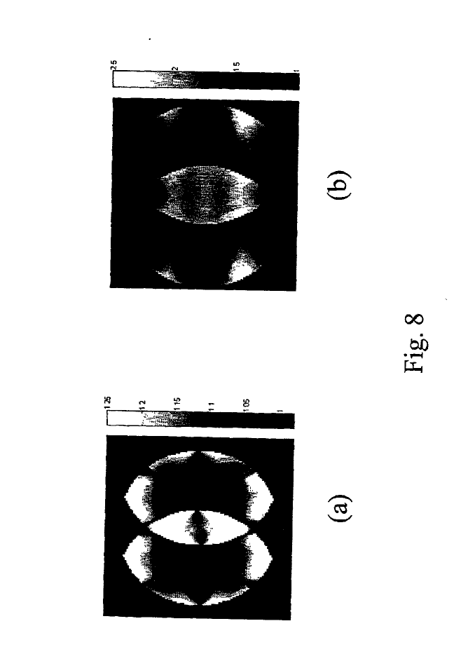

FIG. 8 illustrates simulated g-maps for the head and chest coil portions 102, 104 (again, with 10 mm separation) where a reduction factor of R=3 was simulated. Map (a) shows the head portion simulation, while map (b) shows the chest portion simulation. As can be seen, the g-factor deteriorates at higher reduction factors.

Finally, axial images of a water phantom were acquired using a GE Signa 1.5T MR scanner with 8-channel simultaneous data acquisition. The results were obtained for an axial slice taken at the center of the coil array with an FSE sequence. The images verified the isolation between the neighboring coils, in that there was no demonstrated energy transfer between neighboring loops even though all eight coils were simultaneously receiving NMR RF signals. The successful implementation of inductors for decoupling validates that the present design is a viable alternative to the overlapped coil design, and thus may be used for sensitivity-based reconstruction techniques. Empty Q factors (350→270) are affected by the implementation of the transformers, but without any effect on loaded Q factors.

Through the use of the above-described array coil configuration, a faster, parallel imaging technique such as SENSE may be incorporated into MR imaging with improved SNR. Although there is an inherent increase in coil coupling due to the geometric separation of the individual coil elements, the improved g factor (for a reduction factor R=2) results in an overall higher SNR when using SENSE as compared to a conventional array coil.

While the invention has been described with reference to a preferred embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

1. A multiple channel array coil for magnetic resonance imaging, comprising:
a cylindrically tapered head portion, said head portion including a plurality of individual coil elements; and
a chest portion, said chest portion further comprising a generally planar anterior section and a generally planar posterior section, both said anterior section and said posterior section including a plurality of individual coil elements.

2. The array coil of claim 1, wherein:
each of said plurality of individual coil elements within said head portion are geometrically spaced apart from an adjacent coil element thereto in a non-overlapping configuration; and
each of said plurality of individual coil elements within said chest portion are geometrically spaced apart from an adjacent coil element thereto in a non-overlapping configuration.

3. The array coil of claim 2, wherein each of said plurality of individual coil elements within said head portion and said chest portion are isolated from nearest neighbor coil elements by transformer decoupling.

4. The array coil of claim 3, wherein each of said plurality of individual coil elements is isolated from next-nearest neighbor coil elements by preamplifier decoupling.

5. The array coil of claim 1, further comprising 8 individual coil elements within said head portion and 8 individual coil elements within said chest portion.

6. A multiple channel array coil for magnetic resonance imaging, comprising:
a cylindrically tapered head portion, said head portion including a plurality of individual coil elements;
a chest portion, said chest portion further comprising a generally planar anterior section and a generally planar posterior section, both said anterior section and said posterior section including a plurality of individual coil elements; and
a hinge assembly, said hinge assembly enabling said anterior section of said chest portion to be rotated about a left-right axis and translated in a vertical axis of the array coil.

7. The array coil of claim 6, wherein:
each of said plurality of individual coil elements within said head portion are geometrically spaced apart from an adjacent coil element thereto in a non-overlapping configuration; and
each of said plurality of individual coil elements within said chest portion are geometrically spaced apart from an adjacent coil element thereto in a non-overlapping configuration.

8. The array coil of claim 7, wherein each of said plurality of individual coil elements within said head portion and said chest portion are isolated from nearest neighbor coil elements by transformer decoupling.

9. The array coil of claim 8, wherein each of said plurality of individual coil elements is isolated from next-nearest neighbor coil elements by preamplifier decoupling.

10. The array coil of claim 6, further comprising 8 individual coil elements within said head portion and 8 individual coil elements within said chest portion.

11. A magnetic resonance imaging (MRI) system, comprising:
a computer;
a magnet assembly for generating a polarizing magnetic field;
a gradient coil assembly for applying gradient waveforms to said polarizing magnetic field along selected gradient axes; and
a radio frequency (RF) transceiver system for applying RF energy to excite nuclear spins of an object to be imaged, and for thereafter detecting signals generated by excited nuclei of said object to be imaged, said RF transceiver system further comprising:
a multiple channel array coil having a cylindrically tapered head portion and a chest portion;
said head portion including a plurality of individual coil elements; and
said chest portion further comprising a generally planar anterior section and a generally planar posterior section, both said anterior section and said posterior section including a plurality of individual coil elements;
wherein signals detected by said multiple channel array coil are processed by said computer to produce MR images of said object to be imaged.

12. The MRI system of claim 11, wherein said multiple channel array coil is configured for sensitivity encoding (SENSE) imaging techniques.

13. The MRI system of claim 11, wherein:
each of said plurality of individual coil elements within said head portion are geometrically spaced apart from an adjacent coil element thereto in a non-overlapping configuration; and
each of said plurality of individual coil elements within said chest portion are geometrically spaced apart from an adjacent coil element thereto in a non-overlapping configuration.

14. The MRI system of claim 13, wherein each of said plurality of individual coil elements within said head portion and said chest portion are isolated from nearest neighbor coil elements by transformer decoupling.

15. The MRI system of claim 14, wherein each of said plurality of individual coil elements within said head portion and said chest portion is isolated from next-nearest neighbor coil elements by preamplifier decoupling.

16. The MRI system of claim 11, further comprising 8 individual coil elements within said head portion and 8 individual coil elements within said chest portion.

17. A method for configuring a multiple channel array coil suitable for use in sensitivity encoding for magnetic resonance imaging (MRI), the method comprising:
arranging a first set of individual coil elements into a cylindrically tapered head portion; and
arranging a second and a third set of individual coil elements into a chest portion, said chest portion further comprising a generally planar anterior section including said second set of individual coil elements and a generally planar posterior section including said third set of individual coil elements.

18. The method of claim 17, wherein:
each of first set of individual coil elements within said head portion are geometrically spaced apart from an adjacent coil element thereto in a non-overlapping configuration; and
each of said second and third sets of individual coil elements within said chest portion are geometrically spaced apart from an adjacent coil element thereto in a non-overlapping configuration.

19. The method of claim 13, further comprising isolating each individual coil element in said first, second and third sets from nearest neighbor coil elements by transformer decoupling.

20. The method of claim 19, further comprising isolating each individual coil element in said first, second and third sets from next-nearest neighbor coil elements by preamplifier decoupling.

21. The method of claim 17, further comprising arranging 8 individual coil elements within said head portion, and 4 individual coils within both said anterior and posterior sections of said chest portion.

22. A method for implementing sensitivity encoding for magnetic resonance imaging (MRI), the method comprising:
generating a polarizing magnetic field;
applying gradient waveforms to said polarizing magnetic field along selected gradient axes; and
applying RF energy generated by an RF transceiver system to excite nuclear spins of an object to be imaged, and thereafter detecting signals generated by excited nuclei of said object to be imaged, wherein said RF transceiver system further includes:
a first set of individual coil elements arranged into a cylindrically tapered head portion; and
a second and a third set of individual coil elements arranged into a chest portion, said chest portion further comprising a generally planar anterior section including said second set of individual coil elements and a generally planar posterior section including said third set of individual coil elements.

23. The method of claim 22, wherein:
each of first set of individual coil elements within said head portion are geometrically spaced apart from an adjacent coil element thereto in a non-overlapping configuration; and
each of said second and third sets of individual coil elements within said chest portion are geometrically spaced apart from an adjacent coil element thereto in a non-overlapping configuration.

24. The method of claim 23, further comprising isolating each individual coil element in said first, second and third sets from nearest neighbor coil elements by transformer decoupling.

25. The method of claim 23, further comprising isolating each individual coil element in said first, second and third sets from next-nearest neighbor coil elements by preamplifier decoupling.
The method of claim 22, wherein said RF transceiver system further includes a 16-channel, neurovascular array coil.
